(19) United States
(12) Patent Application Publication
(10) Pub. No.: US 2009/0132013 A1
(43) Pub. Date: May 21, 2009

(54) Cooling Medical Device

(76) Inventor: Michael Amalfi, Pittsford, NY (US)

Correspondence Address: HODGSON RUSS LLP;THE GUARANTY BUILDING, 140 PEARL STREET, SUITE 100, BUFFALO, NY, 14202-4040, US

(21) Appl. No.: 12/235,339

(22) Filed: Sep. 22, 2008

Related U.S. Application Data
(60) Provisional application No. 60/974,244, filed on Sep. 21, 2007.

(51) Int. Cl. A61F 7/10; A61G 15/00

(52) U.S. Cl. 607/96; 62/529; 128/845

(57) ABSTRACT

A medical device embodied as a stretcher, body bag, or mattress is disclosed. The device includes water and a selectively breakable package. The selectively breakable package includes a chemical, such as ammonium nitrate, which when mixed with water will cause an endothermic reaction. In use, the package is broken in order to cause the water to cool. The cooled water absorbs heat from an injured person placed on the device, thereby minimizing inflammation that may occur from injuries sustained by the person.

### CROSS-REFERENCE TO RELATED APPLICATIONS

This application claims priority to U.S. patent application Ser. No. 60/974,244, filed on Sep. 21, 2007, the disclosure of which is incorporated herein by reference.

### FIELD OF THE INVENTION

This invention relates to the field of emergency medical devices, including the use of cooling devices embedded within medical equipment.

### BACKGROUND OF THE INVENTION

Great interest is currently being generated in the field of spinal cord injury, heart attack, stroke, and other traumatic injury treatment by the use of cooling to control swelling and prevent further injury. The efficacy of this treatment increases as the time between initial injury and application of the treatment decreases. Therefore, it is desirable to have equipment for use at the scene of the injury which will enable such treatment.

Also, great interest exists in the area of response to natural disasters. Currently, body bags are used for the storage and transportation of human remains. The decomposition of those remains hinders efforts to determine the identity and cause of death. Decomposition is aided by heat, and therefore it is desirable to have body bags which include a cooling device.

### BRIEF SUMMARY OF THE INVENTION

A medical device according to the invention includes one or more cooling devices. The cooling device may be imbedded within medical equipment. Alternatively, the cooling device may be fitted into a patient support device, or otherwise applied to a patient support surface. Such medical devices may be used to prevent swelling in an injured person or to preserve the remains of a dead person. In an embodiment of the invention, the medical device may be a litter or stretcher having a mattress, which includes an outer bag and a selectively breakable inner bag. The outer bag may include water and the inner bag may include a chemical compound which will react with the water to produce an endothermic reaction. Alternatively, the inner bag may include water and the outer bag may include the chemical compound.

In one embodiment of the invention, the cooling assembly may include an impermeable package, which may be filled with water, and the impermeable package may enclose a selectively breakable package. The selectively breakable package may contain a chemical compound. In another embodiment of the invention, the impermeable package may include the chemical compound and the selectively breakable package may contain water. The chemical compound (“reactant”), such as ammonium nitrate, reacts with water to produce an endothermic reaction. This assembly is referred to herein as the “cooling assembly.” In use, the selectively breakable package is broken in order to allow the reactant to react with the water thereby causing an endothermic reaction. This reaction will cool the water contained within the impermeable package, the package itself, and any objects in thermal communication with the package.

The present invention may be directed to a medical device for supporting a human body. The medical device may be used for transportation of an injured person or a dead body. In this use, the cooling assembly may form, may be embedded within, or may be placed upon a mattress upon which the injured person is placed. The cooling assembly may be adapted to allow the initiation of the reaction before or after placement of the injured person. The mattress may be adapted to fit an emergency gurney, litter or stretcher of the types known in the art. Multiple cooling assemblies may be used in this device.

The present invention may be directed to a medical bed for the support of an injured person. In this use, the cooling assembly may form, may be embedded within, or may encapsulate a mattress upon which the injured person is placed. The mattress may be adapted to fit a hospital bed, which may include elevating and reclining features, of the type known in the art. Multiple cooling assemblies may be used in this device.

The present invention may be directed to a device for the storage and transportation of human remains. In this device, the cooling assembly may form any or all of the surfaces of a body bag. Multiple cooling assemblies may be used to form the bag. The body bag may also include an outer insulating layer.

### BRIEF DESCRIPTION OF THE DRAWINGS

The invention will be described further by way of non-limiting examples, with reference to the attached drawings and diagrams in which:

FIG. 1a is a perspective view of a cooling assembly in the form of a gurney mattress pad according to the invention and a stretcher;

FIG. 1b is a perspective view of a gurney with a mattress having an embedded cooling assembly according to the invention;

FIG. 1c is a perspective view of a gurney with a cooling assembly in the form of a mattress that is in keeping with the invention;

FIG. 1d is a perspective view of a cooling assembly in the form of a gurney mattress pad according to an alternate embodiment of the invention and a stretcher;

FIG. 1e is a perspective view of a gurney with a mattress having an embedded cooling assembly according to an alternate embodiment of the invention;

FIG. 1f is a perspective view of a gurney with a cooling assembly in the form of a mattress according to an alternate embodiment of the invention;

FIG. 2a is a perspective view of a litter wherein the supporting surface is made from a cooling assembly that is in keeping with the invention;

FIG. 2b is a perspective view of a litter wherein the supporting surface is made from a cooling assembly according to an alternate embodiment of the invention;

FIG. 3a is a side view of a cooling assembly in the form of a mattress pad according to the invention and a hospital bed;

FIG. 3b is a side view of a hospital bed with a mattress having embedded multiple cooling assemblies that are in keeping with the invention;

FIG. 3c is a side view of a hospital bed having a cooling assembly embedded within a mattress;

FIG. 3d is a side view of a cooling assembly in the form of a mattress pad according to an alternate embodiment of the invention and a hospital bed;

FIG. 3e is a side view of a hospital bed with a mattress having embedded multiple cooling assemblies according to an alternate embodiment of the invention;

FIG. 3f is a side view of a hospital bed having a cooling assembly embedded within a mattress according to an alternate embodiment of the invention;

FIG. 4a is a side view of a body bag wherein a cooling assembly is formed into a bottom surface of the body bag; and

FIG. 4b is a side view of a body bag wherein a cooling assembly is formed into a top surface of the body bag;

FIG. 4c is a side view of a body bag wherein a cooling assembly is formed into a bottom surface of the body bag according to an alternate embodiment of the invention; and

FIG. 4d is a side view of a body bag wherein a cooling assembly is formed into a top surface of the body bag according to an alternate embodiment of the invention.

### DETAILED DESCRIPTION OF THE INVENTION

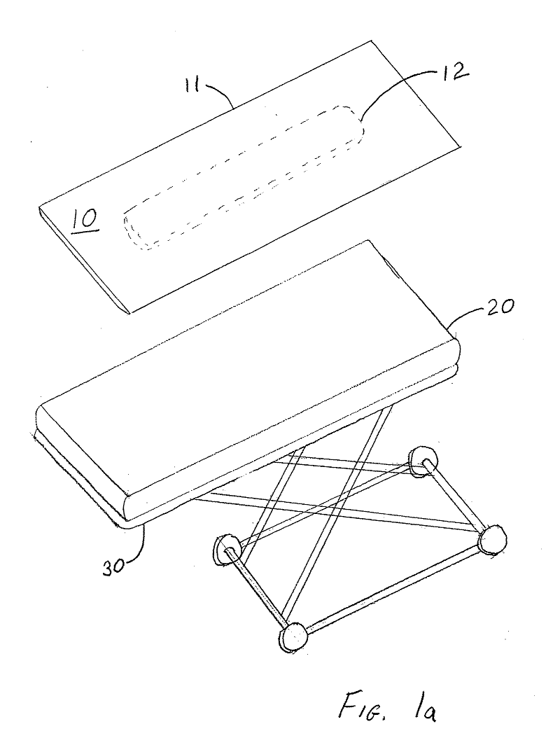

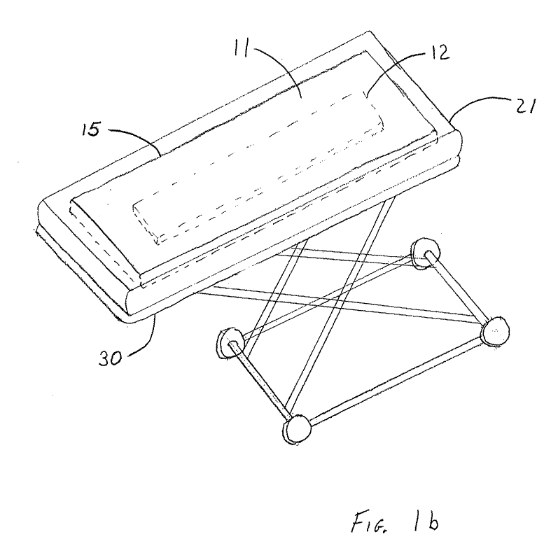

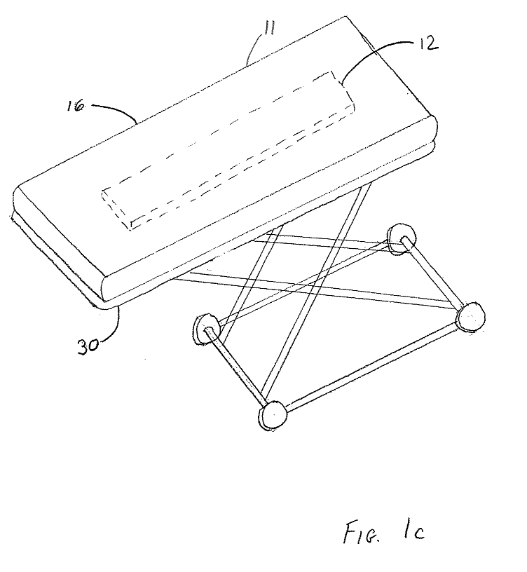

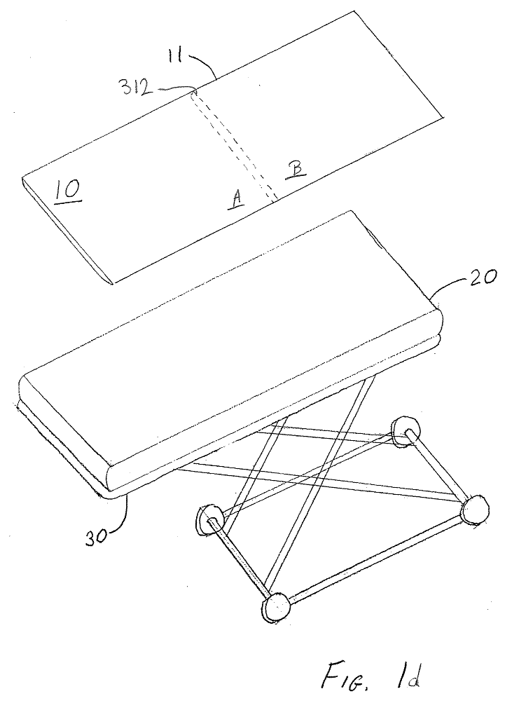

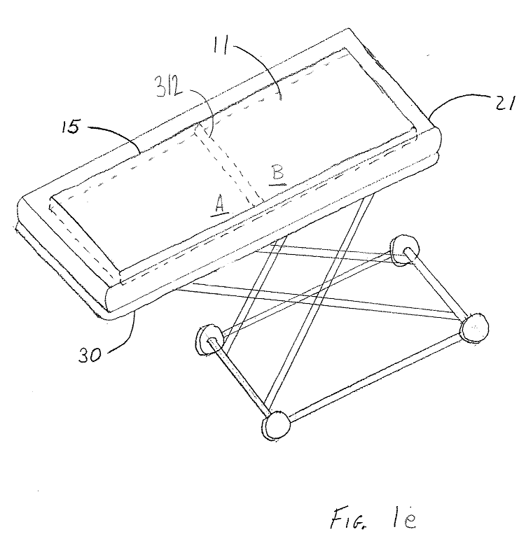

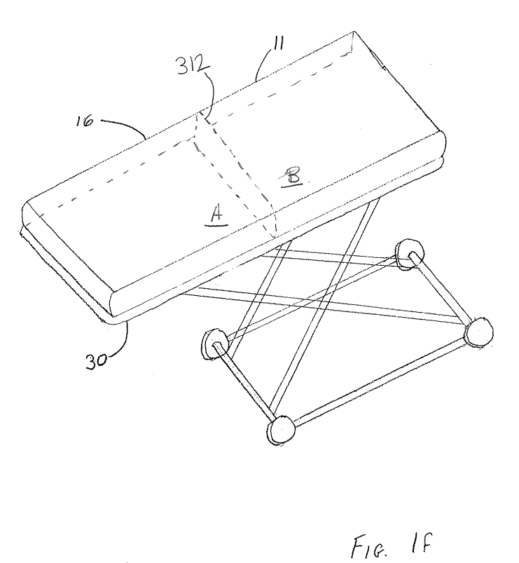

FIGS. 1a-1f show embodiments of the invention wherein the cooling assembly is adapted for use with a gurney 30. FIG. 1a shows a cooling assembly 10 including an impermeable package 11 having water therein. The impermeable package 11 may be constructed of a rigid material or a flexible material, as required for the specific application in which the cooling assembly 10 is to be used. A suitable rigid material may be a plastic material. A suitable flexible material may be a rubber or vinyl material. The impermeable package 11 may be dimensioned to fit onto a mattress 20 of the gurney 30. The impermeable package 11 may be selectively removable from the mattress 20 and the gurney 30. Sealed within the impermeable package 11 may be a selectively breakable package 12. Sealed within the selectively breakable package 12 may be a reactant.

For simplicity, the invention is described herein where the impermeable package includes water and the selectively breakable package includes the reactant. However, it should be understood that the impermeable package may include the reactant and the selectively breakable package may include the water.

FIG. 1b shows a cooling assembly 15 embedded into the top surface of a gurney mattress 21. The impermeable package 11 may be dimensioned to fit into the gurney mattress 21. Sealed within the impermeable package 11 may be the selectively breakable package 12. Sealed within the selectively breakable package 12 may be a reactant. The entire cooling assembly 15 may therefore be contained within the top surface of the gurney mattress 21.

FIG. 1c shows a cooling assembly 16 formed into a mattress shape, wherein the cooling assembly 16 replaces the mattress placed on the gurney 30. In this example, the entire mattress may be viewed as the impermeable package 11. Sealed within the impermeable package 11 may be the selectively breakable package 12. Sealed within the selectively breakable package 12 may be a reactant.

FIG. 1d shows an alternate embodiment of the cooling assembly 10. In this embodiment of the invention, the impermeable package 11 includes a selectively breakable barrier 312 formed in the interior of the impermeable package 11, thereby forming two chambers A, B within the impermeable package 11. Water may be included in one of the chambers A, B and a reactant may be included in the other chamber A, B.

FIG. 1e shows an alternate embodiment of the cooling assembly 15. In this embodiment of the invention, the impermeable package 11 includes a selectively breakable barrier 31 formed in the interior of the impermeable package 11, thereby forming two chambers A, B within the impermeable package 11. Water may be included in one of the chambers A, B and a reactant may be included in the other chamber A, B.

FIG. 1f shows an alternate embodiment of the cooling assembly 16. In this embodiment of the invention, the impermeable package 11 includes a selectively breakable barrier 312 formed in the interior of the impermeable package 11, thereby forming two chambers A, B within the impermeable package 11. Water may be included in one of the chambers A, B and a reactant may be included in the other chamber A, B.

In each of the above embodiments of the invention, the reactant may be ammonium nitrate, which will react with water to produce an endothermic reaction. In use, the selectively breakable package 12 or the selectively breakable barrier 312 is broken in order to allow the reactant to react with the water contained within the impermeable package 11, thereby causing an endothermic reaction. This reaction will cool the water contained within the impermeable package 11, the impermeable package 11 itself, and any objects in thermal communication with the package, including a person's body. The selectively breakable package 12 or the selectively breakable barrier 312 may be broken before a person's body is placed on the cooling assembly. Alternatively, the selectively breakable package 12 or the selectively breakable barrier 312 may be broken after a person's body is placed on the cooling assembly, or at the same time a person's body is placed on the cooling assembly. One method of breaking the selectively breakable package 12 or the selectively breakable barrier 312, in order to allow the reactant to mix with the water, is to apply pressure to the selectively breakable package 12 or the selectively breakable barrier 312. Pressure may be applied to the selectively breakable package 12 or the selectively breakable barrier 312 by placement of the body on the impermeable package 11 or by force applied to the impermeable package 11 by a user, such as a doctor, nurse, EMT, etc.

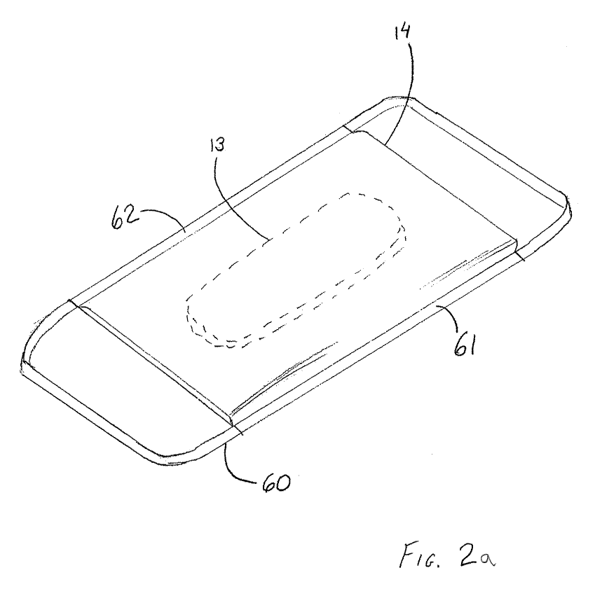

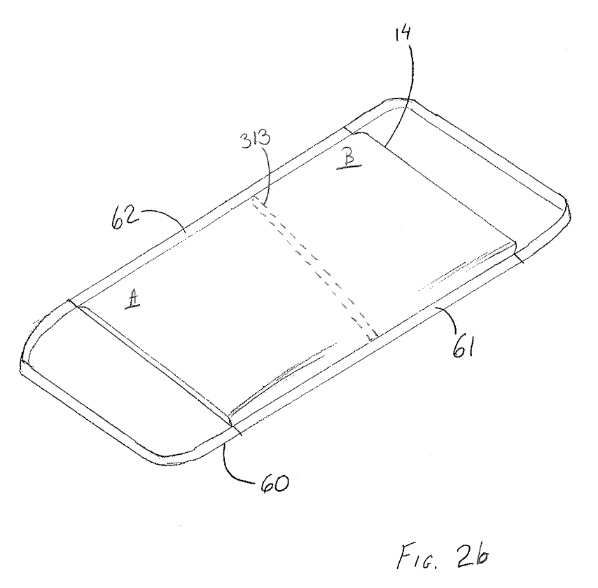

FIG. 2a shows another embodiment of the invention wherein the cooling assembly is adapted for use with a litter 60. In this embodiment, an impermeable package 14 is made to fit between the side rails 61 and 62 of the litter 60. Sealed within the impermeable package 14 is a selectively breakable package 13. Sealed within the selectively breakable package 13 is the reactant. In use, the selectively breakable package 13 may be broken before, after, or in synchronization with a person's body being placed on the cooling assembly, in order to allow the reactant to react with the water contained within the impermeable package 14 thereby causing an endothermic reaction. This reaction will cool the water contained within the impermeable package 14, the impermeable package 14 itself, and any objects in thermal communication with the package, including a person's body.

FIG. 2b shows an alternate embodiment of the cooling assembly shown in FIG. 2a. In this embodiment of the invention, the impermeable package 14 includes a selectively breakable barrier 313 formed in the interior of the impermeable package 14, thereby forming two chambers A, B within the impermeable package 14. Water may be included in one of the chambers A, B and a reactant may be included in the other chamber A, B.

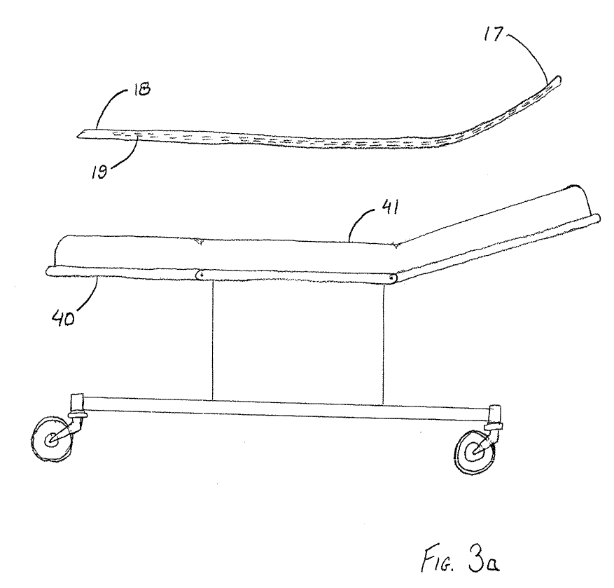

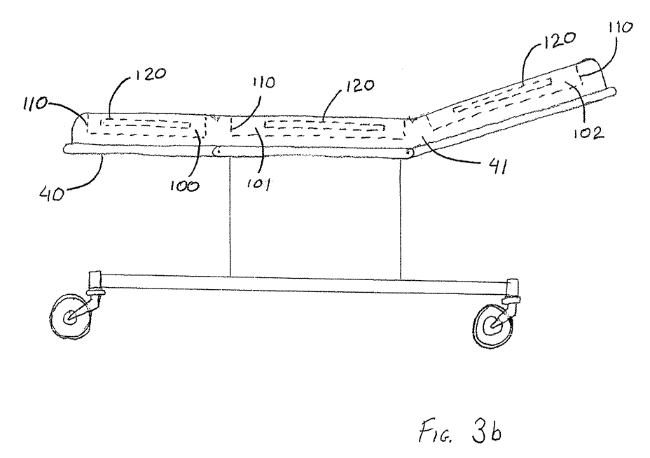

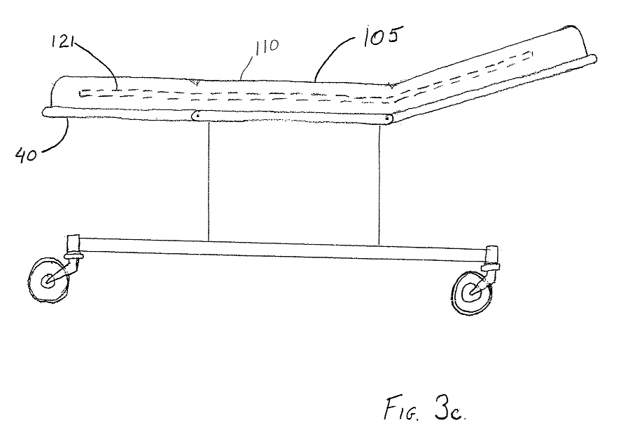

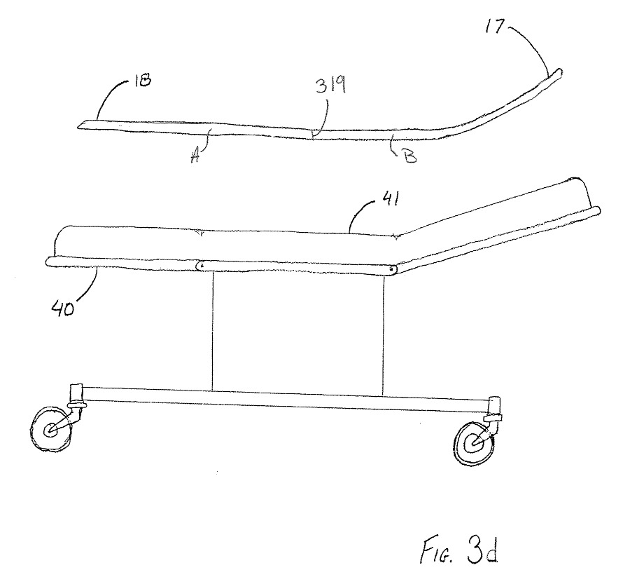

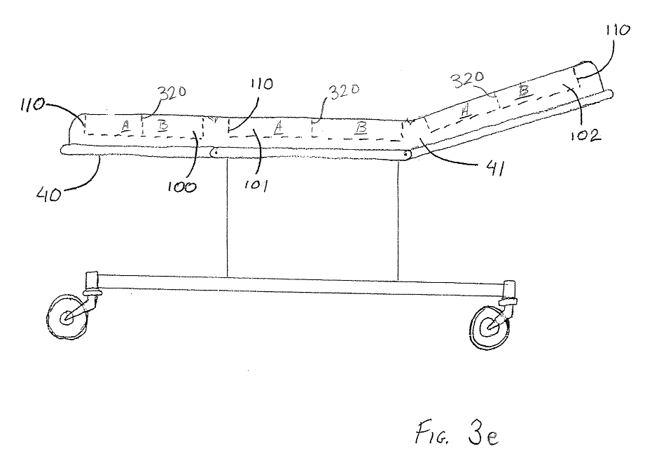

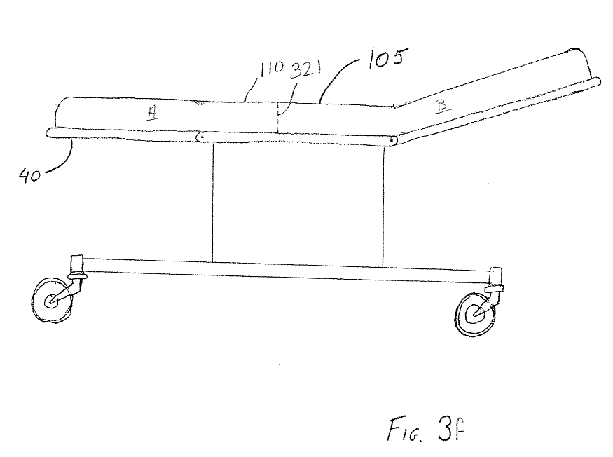

FIGS. 3a-3f show embodiments of the invention wherein the cooling assembly is adapted for use with a hospital bed 40. FIG. 3a shows a cooling assembly 17 including an impermeable package 18 having water therein. The impermeable package 18 may be dimensioned to fit onto a mattress 41 of the hospital bed 40. Sealed within the impermeable package 18 is a selectively breakable package 19. Sealed within the selectively breakable package 19 is a reactant.

FIG. 3b shows multiple cooling assemblies 100, 101, and 102 embedded into the mattress 41. Each cooling assembly 100, 101, and 102 includes an impermeable package 110 having water therein. Sealed within each impermeable package 110 is a selectively breakable package 120. Sealed within each selectively breakable package 120 is a reactant.

FIG. 3c shows a cooling assembly 105 formed into a mattress shape, wherein the cooling assembly 105 replaces the mattress 41 of the hospital bed 40. In this example, the entire mattress 41 may be viewed as the impermeable package 110. Sealed within the impermeable package 110 is a selectively breakable package 121. Sealed within the selectively breakable package 121 is a reactant.

FIG. 3d shows an alternate embodiment of the cooling assembly 17. In this embodiment of the invention, the impermeable package 18 includes a selectively breakable barrier 319 formed in the interior of the impermeable package 18, thereby forming two chambers A, B within the impermeable package 18. Water may be included in one of the chambers A, B and a reactant may be included in the other chamber A, B.

FIG. 3e shows an alternate embodiment of the cooling assemblies 100, 101, 102. In this embodiment of the invention, the impermeable package 110 includes a selectively breakable barrier 320 formed in the interior of the impermeable package 110, thereby forming two chambers A, B within the impermeable package 110. Water may be included in one of the chambers A, B and a reactant may be included in the other chamber A, B.

FIG. 3f shows an alternate embodiment of the cooling assembly 105. In this embodiment of the invention, the impermeable package 110 includes a selectively breakable barrier 321 formed in the interior of the impermeable package 110, thereby forming two chambers A, B within the impermeable package 110. Water may be included in one of the chambers A, B and a reactant may be included in the other chamber A, B.

In each of the above embodiments of the invention, the reactant may be ammonium nitrate, which will react with water to produce an endothermic reaction. In use, the selectively breakable package 12, 13, 19, 120, 121, or the selectively breakable barrier 312, 313, 319, 320, 321 is broken in order to allow the reactant to react with the water contained within the impermeable package 11, 14, 18, 110 thereby causing an endothermic reaction. This reaction will cool the water contained within the impermeable package 14, 18, 110, the impermeable package itself, and any objects in thermal communication with the package, including a person's body.

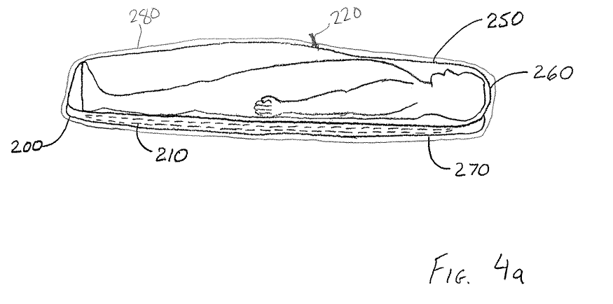

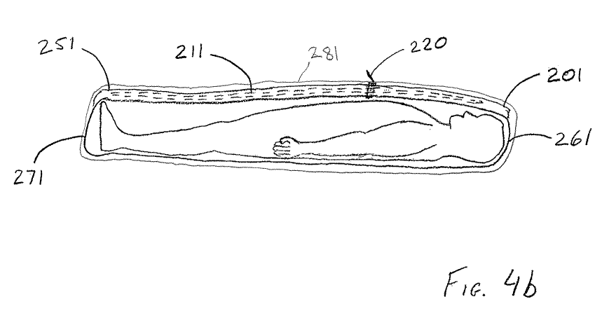

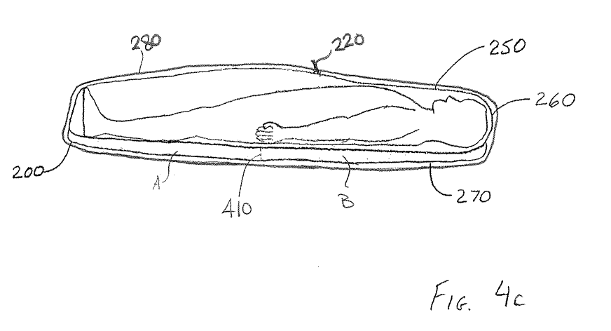

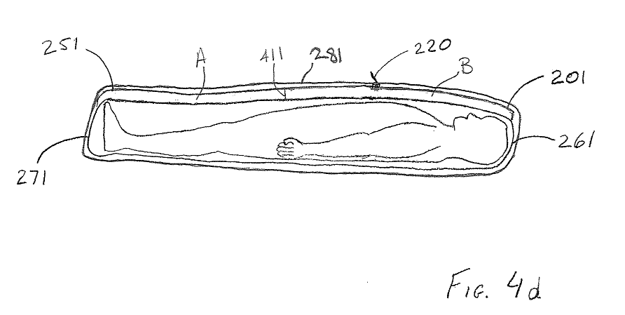

FIGS. 4a-4d show different embodiments of the invention wherein the cooling assembly is adapted for use with a body bag. FIG. 4a shows a body bag 260 having a bottom surface 270 and a top surface 250. In this embodiment, the bottom surface 270 is formed by the impermeable package 200. The impermeable package 200 has water therein. A selectively breakable package 210 is sealed within the impermeable package 200. A reactant is sealed within the selectively breakable package 210. The impermeable package 200 may be adapted to accommodate a zipper 220 or other type of resealable closure to allow insertion or removal of a body. The body bag 260 may also include an outer insulating layer 280 to assist with cooling by reducing the amount of ambient heat allowed to penetrate into the body bag 260. The insulating layer 280 may be constructed from any known light-weight insulating material such as a black scrim PVC having a liner, or a heavy duty vinyl PVC, as known in the art.

FIG. 4b shows a body bag 261 having a bottom surface 271 and a top surface 251. In this embodiment, the top surface 251 of the body bag 261 is formed by the impermeable package 201. The impermeable package 201 has water therein. A selectively breakable package 211 is sealed within the impermeable package 201. A reactant is sealed within the selectively breakable package 211. The impermeable package 201 may be adapted to accommodate a zipper 220 or other type of resealable closure to allow insertion or removal of a body. The body bag 261 may also include an outer insulating layer 281 to assist with cooling by reducing the amount of ambient heat allowed to penetrate into the body bag 261. The insulating layer 281 may be constructed from any known insulating material.

FIG. 4c shows an alternate embodiment of the cooling assembly. In this embodiment of the invention, the impermeable package 200 includes a selectively breakable barrier 410 formed in the interior of the impermeable package 200, thereby forming two chambers A, B within the impermeable package 200. Water may be included in one of the chambers A, B and a reactant may be included in the other chamber A, B.

FIG. 4d shows an alternate embodiment of the cooling assembly. In this embodiment of the invention, the impermeable package 201 includes a selectively breakable barrier 411 formed in the interior of the impermeable package 201, thereby forming two chambers A, B within the impermeable package 201. Water may be included in one of the chambers A, B and a reactant may be included in the other chamber A, B.

In each of the above embodiments of the invention, the reactant may be ammonium nitrate, which will react with water to produce an endothermic reaction. In use, the selectively breakable package 210, 211 or the selectively breakable barrier 410, 411 is broken in order to allow the reactant to react with the water contained within the impermeable package 200, 201 thereby causing an endothermic reaction. This reaction will cool the water contained within the impermeable package 200, 201, the impermeable package itself, and any objects in thermal communication with the package, including a person's body.

Although embodiments of the invention have been described herein, the invention is not limited to such embodiments. The claims which follow are directed to the invention, and are intended to further describe the invention, but are not intended to limit the scope of the invention.

What is claimed is:

1. A body support device having a patient support surface, comprising:
an impermeable package in a shape adapted to removably fit on the patient support surface;
water enclosed within the impermeable package;
a selectively breakable package enclosed within the impermeable package; and
reactant enclosed within the selectively breakable package.

2. The device of claim 1 wherein the patient support surface is a litter.

3. The device of claim 1 wherein the patient support surface is a gurney.

4. The device of claim 1 wherein the patient support surface is a hospital bed.

5. The device of claim 1 wherein the impermeable package is a mattress adapted for use on a gurney or hospital bed.

6. The device of claim 1 wherein the reactant is ammonium nitrate.

7. The device of claim 1 wherein the impermeable package is made from a rigid material.

8. The device of claim 1 wherein the impermeable package is made from a flexible material.

9. A body support device having a patient support surface, comprising:
an impermeable package in a shape adapted to removably fit on the patient support surface;
reactant enclosed within the impermeable package;
a selectively breakable package enclosed within the impermeable package; and
water enclosed within the selectively breakable package.

10. The device of claim 9 wherein the patient support surface is a litter.

11. The device of claim 9 wherein the patient support surface is a gurney.

12. The device of claim 9 wherein the patient support surface is a hospital bed.

13. The device of claim 9 wherein the impermeable package is a mattress adapted for use on a gurney or hospital bed.

14. The device of claim 9 wherein the reactant is ammonium nitrate.

15. The device of claim 9 wherein the impermeable package is made from a rigid material.

16. The device of claim 9 wherein the impermeable package is made from a flexible material.

17. A device for use in preserving human remains, comprising a body bag having:
a bottom surface comprising:
an impermeable package;
water enclosed within the impermeable package;
a selectively breakable package enclosed within the impermeable package; and
reactant enclosed within the selectively breakable package.

18. The device of claim 17, further comprising an insulating outer layer.

19. A device for use in preserving human remains, comprising a body bag having:
a top surface comprising:
an impermeable package;
water enclosed within the impermeable package;
a selectively breakable package enclosed within the impermeable package; and
reactant enclosed within the selectively breakable package.

20. The device of claim 19, further comprising an insulating outer layer.

21. A body support device having a patient support surface, comprising:
an impermeable package in a shape adapted to removably fit on the patient support surface;
a selectively breakable barrier formed within the impermeable package, wherein the selectively breakable barrier defines at least two chambers within the impermeable package;
water included within a first one of the chambers; and
a reactant included within a second one of the chamber of the impermeable package.

22. The device of claim 21 wherein the patient support surface is a litter.

23. The device of claim 21 wherein the patient support surface is a gurney.

24. The device of claim 21 wherein the patient support surface is a hospital bed.

25. The device of claim 21 wherein the impermeable package is a mattress adapted for use on a gurney or hospital bed.

26. The device of claim 21 wherein the reactant is ammonium nitrate.

27. The device of claim 21 wherein the impermeable package is made from a rigid material.

28. The device of claim 21 wherein the impermeable package is made from a flexible material.
